(19) United States
(12) Patent Application Publication
(10) Pub. No.: US 2009/0124699 A1
(43) Pub. Date: May 14, 2009

(54) Silicone Material for Releasing an Active Molecule

(75) Inventors: Helene Lannibois, Charenton Le Pont (FR); Christian Pusineri, Serezin Du Rhone (FR)

Correspondence Address: BUCHANAN, INGERSOLL & ROONEY PC, POST OFFICE BOX 1404, ALEXANDRIA, VA, 22313-1404, US

(73) Assignee: BLUESTAR SILICONES FRANCE SAS, Lyon (FR)

(21) Appl. No.: 11/794,094

(22) PCT Filed: Dec. 23, 2005
(86) PCT No.: PCT/FR05/03271

§ 371 Date: Jun. 12, 2008

(30) Foreign Application Priority Data
Dec. 24, 2004 (FR) 0413931

(51) Int. Cl. A61K 47/24; A61K 47/30; A61K 31/045; A61K 31/16

(52) U.S. Cl. 514/625; 514/772; 514/785; 514/772.3; 514/724

(57) ABSTRACT

The invention concerns a material, preferably adhesive, for releasing an active molecule for cosmetic use or personal care or a pharmaceutically or biologically active molecule for pharmaceutical use, which is formed of a silicone substance, preferably adhesive, wherein is incorporated said molecule and a compatibility agent wherein said active molecule is soluble, said compatibility agent being selected among isopropyl myristate, isopropyl palmitate, isononyl isononanoate, neopentyl glycol dioctanoate, branched paraffins, organofunctional silicones, or a silicone oil consisting of a cyclic concatenation of 4, 5, 6 or 7 D-siloxyl units of formula: (R)₂SiO_{2/2} formula wherein the symbols R, identical or different, represent each a linear or branched C₁-C₆ alkyl radical, preferably methyl or an alkyl or arylalkyl radical having 6 to 8 carbon atoms, preferably phenyl. The invention also concerns an object incorporating or consisting of such a material.

The present invention relates to a silicone material allowing the release of an active molecule incorporated therein. The present invention concerns, in particular, the release of active molecules which may be used in the pharmaceutical, cosmetic and personal care fields.

In the pharmaceutical and cosmetic fields, these materials are generally known as patches and are intended to be applied to the skin, where they deliver active molecules transdermally or to treat skin disorders.

Silicone patches have been described. These descriptions specify that they are formed by a film of adhesive silicone gel enclosing the active molecule. Silicone gels are biocompatible adhesives which are not aggressive to the skin.

Patent FR 2 618 337 describes a surgical dressing comprising a silicone gel layer associated with a laminated silicone elastomer film. The formulations of the gels and elastomers are polyaddition formulations. The dressing may include active molecules.

Patent FR 2 735 024 also describes a silicone patch. The gel may theoretically contain from 0.01 to 30% of active molecule. However, the contents are limited to a few percent in the examples. Moreover, it is known that the gels consisting of polydimethylsiloxane units together by Si(CH₃)₂—CH₂—CH₂—Si(CH₃)₂— bridges have, as is the case in this document, very limited solvent powers, and this necessarily limits the active molecule content which it is possible to incorporate. Contents from the top of the claimed range (greater than 20% or even than 10%) can in practice not be obtained under satisfactory conditions. The gel described in this patent can also contain a cutaneous transfer promoter intended, as its name indicates, to accelerate the passage of active molecules having a low diffusion rate. It also refers to the possible presence of a solvent, such as ethylene glycol monoethyl ether which is known to promote tissue penetration.

This field presents difficulties for incorporating active molecules. Sufficient contents for an effective application have proven difficult to achieve, especially in the case of an active molecule which is solid at ambient temperature or of an active molecule having low solubility in the formulations of the silicone gels.

The object of the present invention is therefore to propose a new material capable of incorporating active molecule contents which are effective and available for the intended application, i.e. so that the material is capable of incorporating an amount of active molecule compatible with the intended application and of releasing this active molecule, especially on contact with the tissue or the skin for which it is intended.

A further object of the invention is to provide such a material which is adhesive.

The invention therefore relates to a silicone material, preferably an adhesive material, for releasing an active molecule for cosmetic use or in personal care or a pharmaceutically or biologically active molecule for pharmaceutical use, which is formed by a, preferably adhesive, silicone material wherein there is incorporated said molecule and a compatibility agent wherein said active molecule is soluble, said compatibility agent being selected from isopropyl myristate, isopropyl palmitate, isononyl isononanoate, neopentyl glycol dioctanoate, branched paraffins, organofunctional silicones, or else a silicone oil consisting of cyclic chain of 4, 5, 6 or 7 D siloxyl units of formula:

(R)₂SiO_{2/2}

wherein the symbols R, which are the same or different, each represent a linear or branched C₁-C₆ alkyl radical, preferably methyl or an aryl or alkylaryl radical having 6 to 8 carbon atoms, preferably phenyl.

The term “organofunctional silicone” refers to a silicone oil, preferably polydimethylsiloxane, of which some silicon atoms carry organic groups (instead of methyl groups in the case of a polydimethylsiloxane) such as polyethers, paraffins, esters, alcohols, etc., wherein the distribution of these groups can be random or statistical or correspond to a block or comb-shaped copolymer structure. Examples include copolyols of the polydimethylsiloxane/polyether comb-shaped or block copolymer type. These organofunctional silicones are those which are soluble in the silicone material.

The compatibility agent performs various roles and has specific properties. It is pharmaceutically or cosmetically acceptable, i.e. it can be used in contact with the skin without inducing significant toxicity. It is capable of solubilising the active ingredient in substantial proportions and of generating a concentration gradient allowing larger amounts of active material to be released than in the past. Indeed, the solubilised active molecule concentration plays a crucial part in controlling the release kinetics of this active molecule. The compatibility agent is miscible with the silicone components of the silicone material to allow optimum dispersion of the entire incorporated amount of active ingredient in the material. It does not interfere with the crosslinking reaction which is at the origin of the formation of the material. Nevertheless, after crosslinking of the material, it allows the active molecule to diffuse through and beyond the material and, for example, to be delivered on contact with the skin, a tissue, a mucous membrane, etc.

The amount of active molecule incorporated may advantageously exceed the solubilisation capacity of the compatibility agent, so the active molecule is present partially in a solubilised state and partially in a dispersed state (liquid dispersion, for example droplets, or solid dispersion, for example powder). The concentration gradient is established through the material, toward the zone in contact with the skin or the like, and what is known as the dispersed portion is gradually solubilised as the solubilised portion is used up. The dispersed form acts as an active molecule reservoir.

The invention therefore allows the incorporation of higher deliverable active molecule contents than in the past owing to the use of the compatibility agent and moreover, if appropriate, owing to the presence of an active molecule reservoir.

The maximum concentration will vary as a function of the degree of solubility of the active molecule in the material and, in particular, in the compatibility agent and as a function of the content of this agent.

The compatibility agent content may be between 5 and 50% by weight, preferably between 10 and 30% by weight, expressed relative to the total composition.

The concentration of solubilised active material is advantageously at least 1.5 times the maximum concentration which it would be possible to solubilise in the absence of the compatibility agent. It is preferably at least 2 times greater and better still at least 2.5 or 3 times greater. The compatibility agent content may be adjusted so that the amount of solubilised active molecule corresponds to this definition.

However, the total content of active molecule and compatibility agent has to remain within the limits allowing the formation, by crosslinking, of a silicone material having the desired mechanical properties in the knowledge that, in specific cases, it may desirable or useful to place the material in a pocket or the like, as described hereinafter. This total content may be up to 75% by weight of the total composition. More generally, the total content may be up to 50% by weight.

It may also be specified that the concentration of active molecule solubilised in the silicone material may be greater than or equal to 5% by weight based on the total weight of the material. It is preferably greater than or equal to 10% and still more preferably greater than or equal to 15, 20, 25 or 30% by weight based on the total weight of the material (in particular up to 35, 40 or 50% by weight).

The total (solubilised and dispersed) content of active molecule may be between 5 and 60%, preferably between 10 and 40 or 50%, or else between 10 and 30% by weight based on the total weight of the material.

The active ingredient may be mixed with the compatibility agent, then the mixture may be introduced into the composition forming the silicone material or into one of the silicone components thereof.

In a variation, the composition forming the silicone material and the compatibility agent are mixed, then the active molecule is added and mixed.

Obviously, the material may incorporate a plurality of active molecules.

The cosmetic active ingredients which are soluble in the compatibility agent may be selected, in particular, from antioxidants (radical action), vitamins, moisturisers, amino acids, vegetable oils rich in polyunsaturated fatty acids, phytosterols, insaponifiables, ceramides, UV filters, plant extracts, α-hydroxy acids and also additives such as organic pigments.

Examples of medicinal active ingredients soluble in the compatibility agent which can potentially be incorporated in the material according to the invention include, in particular, antibacterial agents, antimycotic agents, antiacne agents, sedatives and tranquillisers, anxiolytics, hormones, androgenic steroids, oestrogenic steroids, progestational steroids, analgesics, hypoglycaemics, antispasmodics, beta blockers, non-steroidal anti-inflammatories, antiosteoporotic agents, cutaneous bleaching agents, vasodilators, antihypertensives, antiparkinsonians, antimigraine agent, anticancer agents and nutrient intakes such as vitamins, essential amino acids and essential fatty acids. Dermatological applications are particularly desirable.

Further examples include compounds having a favourable action in personal care or bodily hygiene, for example a refreshing and/or deodorising or deodorant action, such as menthol and methyl diisopropyl propionamide (WS-23®) supplied by Rhodia.

The silicone material may be a gel or an elastomer. This material is obtained by hydrosilylation reaction (polyaddition) between a polyorganosiloxane carrying alkenylsiloxy units and a polyorganosiloxane carrying hydrogenosiloxy units in the presence of a hydrosilylation catalyst. The crosslinking may be carried out cold or at a low temperature, generally less than 50° C., allowing the integrity of the active molecule to be upheld.

The silicone materials concerned conventionally consist of the product of a hydrosilylation reaction occurring in a mixture comprising basically a polyorganosiloxane carrying reactive alkenyl groups, comprising 2 to 6 carbon atoms, preferably vinyl bound to silicon, a polyorganosiloxane carrying hydrogen atoms bound to silicon, and a platinum catalyst.

Gel formulations have been described in various documents including: U.S. Pat. No. 4,072,635, EP 69 451, EP 322 118, EP 532 362, EP 737 721.

The gels are characterised generally by a penetration value ranging from 150 to 350 tenths of millimetres to standard DIN ISO 2137.

The elastomers to which the invention relates are cold-vulcanisable elastomers (RTV) having a Shore A hardness of between about 5 and about 30 and those having a Shore 00 hardness of between about 15 and about 40 to standard DIN 53505. A person skilled in the art is quite familiar with compositions of this type.

Suitable for carrying out the invention are gel compositions comprising:

(I) at least one polyorganosiloxane POS (I) comprising:
a) M-type terminal siloxyl units of formula:

(R)₂(alkenyl)SiO_{1/2}
wherein the R groups, which are the same or different, are linear or branched C₁-C₆ alkyl groups and/or substituted or unsubstituted aryls, the alkenyl group having preferably 2 to 6 carbon atoms and being more preferably vinyl;
b) D-type siloxyl units, which are same or different,

(R¹)₂SiO_{2/2}
wherein R¹ has the same definition as R,

(II) at least one polyorganosiloxane POS (II) comprising:
a) M-type terminal siloxyl units of formula:

(H)ₛ(R²)ₜSiO_{1/2}
wherein R² has the same definition as R, s is selected from the values 1, 2 and 3 and the sum of s+t is equal to 3
b) D-type siloxyl units, which are the same or different, of formula:

(H)ᵤ(R³)ᵥSiO_{2/2}
wherein R³ has the same definition as R, u is selected from the values 0 and 1, v is selected from the values 1 and 2 and the sum of u+v is equal to 2;
with the condition that at least one of the D units of POS (II) is the carrier of a hydrogen atom (u=1, v=1) and that the sum s+u is greater than or equal to 2;

(III) optionally at least one polyorganosiloxane POS (III) qualified as being an “extender” comprising:
a) D-type siloxyl units of formula:

(R⁵)₂SiO_{2/2};
wherein R⁵ has the same definition as R;
b) M-type terminal siloxyl units of formula:

(H)_{w}(R⁴)xSiO_{1/2}
wherein R⁴ has the same definition as R, w is selected from the values 1, 2 and 3, x is selected from the values 0, 1 and 2 and the sum of w+x is equal to 3;

(IV) optionally at least one polyorganosiloxane POS (IV) which may be used, in particular, as a diluent for POS (I) and comprising M-type terminal siloxyl units of formula:

(R⁶)₃SiO_{1/2}
and D-type siloxyl units of formula:

(R⁷)₂SiO_{2/2}
wherein R⁶, R⁷, which are the same or different, have the same definition as R;

(V) an effective amount of, preferably platinum-type, hydrosilylation catalyst (V).

The ratios of the polyorganosiloxanes POS (I), POS (II) and POS (III) are selected as is known per se for obtaining a gel during the crosslinking process.

In practice, the most readily used forms of POS (I) are polydimethylsiloxanes α, ω (dimethylvinylsiloxy). Such forms of POS (I) are commercially available (for example, RHOEORSIL® 620 V from RHODIA).

POS (II) may comprise H—Si units distributed in the chains and/or at the ends thereof (u=1 or 2). Preferably, these units are located both in the chain and at the chain end.

There are preferably two different types of D units in the forms of POS (II), although it is not ruled out to have as many as are allowed by the combinations u and v of the formula provided hereinbefore for D units of the POS (II).

Examples of POS (II) include:

Poly(dimethylsiloxy) (siloxymethylhydrogeno) α, ω (dimethylhydrogenosiloxy).

These forms of POS (II) are commercially available products such as, for example, RHODORSIL® 626 V 300 H1.7 from RHODIA and have been broadly disclosed in the technical literature with regard to both the structures thereof and the syntheses thereof.

The extender (III) is a polyorganosiloxane (POS) advantageously having H—Si units only on its terminal siloxyl units M. Its viscosity is preferably much lower than that of POS (I), for example of the same order as that of POS (II).

Practical examples of POS (III) include:
Poly(dimethylsiloxy) α, ω (dimethylhydrogenosiloxy).

The structure and process for the preparation of POS (III) usable in the composition of the invention are also broadly illustrated by the prior technical literature. Examples of commercially available products useable as POS (III) include RHODORSIL® 620H2 from RHODIA.

According to an optional but nevertheless advantageous provision of the invention, the POS (I) is diluted using a POS (IV) comprising M and D units wherein the substituents R⁶ and R⁷ are preferably of the same type as the substituents R and R¹ of the POS (I). Even more preferably, R⁶═R⁷═R¹═CH₃. This POS (IV) consists, for example, of a polydimethylsiloxane α, ω (trimethylsiloxy) oil. This type of POS is readily commercially available, for example the product sold by RHODIA under the name RHODORSIL® 47 V 100. The diluent POS (IV) is obviously selected as a function of the nature of POS (I) and it is clear that POS (IV) will by definition be less viscous than POS (I). Thus, according to a preferred feature of the invention, the composition comprises at least a POS (IV) having a basically linear structure and less high dynamic viscosity than POS (I), preferably at least 20 times less high and more preferably still 5 times less high than that of POS (I).

Obviously, the proportions of alkenyl and H—Si groups present in each of POS (I) to (III) of the composition are significant. A non-limiting illustration is provided hereinafter:
POS (I): Vi present in a proportion of from 0.01 to 10% by weight, preferably from 0.05 to 1% by weight and more preferably still in a proportion of approximately 0.1% by weight;
POS (II): H—Si present in a proportion of from 0.01 to 10, preferably from 0.1 to 1.5 and more preferably still in a proportion of approximately 0.7% by weight;
POS (III): H—Si present in a proportion of from 0.01 to 10, preferably from 0.05 to 1 and more preferably still in a proportion of approximately 0.2% by weight.

The catalyst (V) is another important element of the composition according to the invention. It is preferably an organometallic complex of platinum or else one of the platinum-based catalysts traditionally used for catalysing hydrosilylation reactions between SiH radicals and SiVi radicals. Examples include black platinum, chloroplatinic acid, a chloroplatinic acid modified by an alcohol, a complex of chloroplatinic acid with an olefin, an aldehyde, a vinyl siloxane or an acetylenic alcohol. U.S. Pat. No. 2,823,218 discloses a chloroplatinic acid-type hydrosilylation catalyst and U.S. Pat. No. 3,419,593 relates to catalysts formed by vinylsiloxane-type chloroplatinic acid and organosilicone complexes. Platinum and hydrocarbon complexes usable as a hydrosilylation catalyst are disclosed by U.S. Pat. Nos. 3,159,601 and 3,159,662. U.S. Pat. No. 3,723,497 describes a platinum acetylacetonate and U.S. Pat. No. 3,220,972 relates to platinum alcoholate platinum-based catalysts.

With regard to the catalytically effective amounts to be used, it is obvious that a person skilled in the art in the field in question is quite capable of determining the optimum amount of catalyst to promote crosslinking. This entity depends, in particular, on the nature of the catalyst and the forms of POS in question. For the sake of argument, it may be stated that it will be between 0.1 and 40 ppm (for example, 15 ppm) per 100 parts by weight of POS (I).

To pursue other beneficial characteristics of POS (I) to (IV), it may be stated that they advantageously have a substantially linear structure.

The viscosity of the POS of the composition according to the invention is a further parameter for consideration, in particular with the regard to ease of handling of this composition and the viscoelastic properties of the gel obtainable by crosslinking of this composition.

In this regard and according to an advantageous provision of the invention, POS (I) is substantially linear and has a dynamic viscosity of less than or equal to 500,000 mPa·s, preferably between 1,000 and 200,000 mPa·s;

and/or POS (II) is substantially linear and has a dynamic viscosity of less than or equal to 100,000 mPa·s, preferably to 1,000 mPa·s and more preferably still between 10 and 100 mPa·s;

and/or POS (III) is substantially linear and has a dynamic viscosity of less than or equal to 100,000 mPa·s, preferably to 1,000 mPa·s and more preferably still between 10 and 100 mPa·s.

In practice, a composition according to the preferred embodiment of the invention may be characterised in that:
POS (I) comprises M units, up to 0.2 to 1% by weight, wherein R=CH₃, alkenyl=vinyl m=2 and n=1, and also D units, up to 0.9 to 0.98% by weight wherein R¹=CH₃, p=2 and q=0;
POS (II) comprises M units, up to 4 to 6% by weight, wherein R²=CH₃, s=1 and t=2, and D units, up to 12 to 18% by weight, wherein R³—CH₃ or H, v=2 and n=0;
POS (III) comprises M units, up to 8 to 10% by weight, wherein R⁴=CH₃, w=1 and x=2, and D units, up to 80 to 92% by weight, wherein R⁵=CH₃;

and in that there is provided a diluent POS (IV), the units M and D of which respectively comprise R⁶═R⁷═CH₃ and are present up to approximately 10 and 80% by weight respectively, diluent (IV) preferably being present in an amount of less than or equal to 50% by weight, preferably to 40% by weight and more preferably still between 5 and 20% based on the mixture POS (I)+(IV).

The composition of the silicone material may also comprise other ingredients, such as plasticisers and also cutaneous transfer accelerators or adsorption promoters, to promote diffusion of the active ingredient through the skin. These products usually belong to the group of, optionally oxyethylenated, fatty acids, fatty acid esters, fatty alcohols, fatty derivatives of propylene glycol, fatty derivatives of ethylene glycol, fatty derivatives of terpenes. Further examples include ethylene glycol monoethyl ether (for example Transcutol®) which is a well-known diffusion promoter.

The present invention also relates to any item incorporating or consisting of the material according to the invention.

This item may be a patch, preferably an adhesive patch, to be placed in contact with the skin or a tissue, for cosmetic (molecule for cosmetic use) or pharmaceutical (biologically or pharmaceutically active molecule, especially in dermatology) applications.

According to a particular configuration, the patch comprises the material according to the invention, comprising the active molecule, placed on a support allowing application. The material may be present in the form of a film which is a few tenths of millimetres thick, deposited on a support and optionally protected by a protective film.

This item may also be a pocket or the like enclosing the material, formed by a membrane or a film made of a material which is strong but through which the active molecule is able to pass and diffuse. The membrane or film may, for example, be based on polyurethane.

The material according to the invention, which is optionally packaged in a pocket or the like, may be used as an insert, optionally a cushioning insert, in a shoe, a knee pad, a clothing, sports or bodily protection article, and more generally in any item intended to be worn by a living being, in particular a human, at a location in direct or indirect contact with a part of the body, while at the same time having the special properties associated with the diffusion of the active molecule, which is basically of the type of a molecule for personal or cosmetic care, for example menthol, antiperspirants, perfume, deodorant. The invention also relates to any item of this type comprising such an insert.

By the same token, it is also conceivable to impregnate a woven or nonwoven textile article with a material according to the invention, the material being deposited on the textile before crosslinking thereof. The material may also be deposited (for example, a layer ranging from 100 μm to several mm) and fixed to a textile of this type by crosslinking. The invention relates to a textile thus treated and to any article containing such a textile.

With regard to the preparation of the material, it may be stated that the composition is crosslinked at ambient temperature or after heating at temperatures which are not liable to destroy the active molecule and the compatibility agent.

The invention will now be described in greater detail with reference to embodiments taken by way of non-limiting examples.

### EXAMPLE

According to the mode of operation described hereinafter, menthol is solubilised in isopropyl myristate or in D5 or in a mixture of these two solvents. These solutions are then introduced into the mixtures of parts A and B of the gels.

### 2.1. Product Used

Menthol: Merck
Solvents: —pentadimethyl siloxane [D5]=supplier Rhodia Silicones
   isopropyl myristate [IPM]=supplier Merck-Schuchardt
Silicone gel: the gels selected to carry out these tests correspond to bi-component products which crosslink at ambient temperature in a polyaddition reaction in the presence of a platinum catalyst. One of these formulations contains a non-reactive oil which acts as a plasticiser, while the other contains no such oil. The compositions of parts A and B of these two formulations are provided in Table 1.

TABLE 1
compositions of the silicone gel formulations

| | |
|---|---|
| Gel 1 | Gel 2 |

| | Part A-1 | Part B-1 | Part A-2 | Part B-2 |
|---|---|---|---|---|
| Oil 1 | 100 g | 98.2 g | 48.89 g | 48.5 g |
| Oil 2 | — | 1.71 g | 1.16 g | — |
| Oil 3 | — | 0.09 g | 0.06 g | — |
| Catalyst | 0.005 g | — | — | 0.011 g |
| Oil 4 | — | — | 49.89 g | — |

* references of parts A and B of the gels used

The components correspond to the following structures:
oil 1: pdms (polydimethylsiloxane) oil having a viscosity of 60,000 mPa·s with Si—CH═CH2 groups located at chain ends; CH═CH₂ content ˜0.06 at 0.1% by weight;
oil 2: pdms oil having a viscosity of 300 mPa·s with a CH₃SiH and Si(CH₃)₂H group; H content ˜0.17% by weight;
oil 3: pdms oil having a viscosity of 7 to 10 mPa·s with Si(CH₃)₂H groups located at chain ends; H content ˜0.19% by weight;
oil 4: polydimethylsiloxane oil without reactive groups having a viscosity of 100 mPa·s;
10% platinum catalyst having a degree of oxidation of 0.

### 2.2. Menthol Solutions

The menthol solutions were prepared by dissolving menthol crystals in D5 or IPM at 30-50° C. Once they had dissolved, the solutions were brought back to ambient temperature; the second solvent was added at this time if appropriate.

The characteristics of the solutions used are set out in the following Table 2.

TABLE 2
reference and composition of the menthol
solutions: in % by weight.

| | IPM | D5 | Menthol |
|---|---|---|---|
| Solution A | 50 | — | 50 |
| Solution B | 25 | 25 | 50 |

### 3. Menthol Patches

The mixtures of the various components used to prepare the patches were obtained at ambient temperature as follows:
parts A and B of each of the gel formulations were mixed by hand using a spatula in a proportion of 1 part of A to 1 part of B;
the various amounts of solutions and solvents were weighed then introduced—again as a result of being mixed by hand—into the preceding mixtures;
the mixtures were then degassed so as to eliminate the air bubbles introduced at the time the mixtures were prepared. The degassing was carried out by treatment under slight vacuum.

As soon as they had been prepared, the mixtures were poured into Petri dishes or polyethylene boxes. The crosslinkings were carried out at ambient temperature over 12 to 24 hours.

The characteristics of the patches prepared using this mode of operation are set out in Table 3.

TABLE 3
characteristics of the menthol patches

| | | | | | | | PATCH | |
|---|---|---|---|---|---|---|---|---|
| | MENTHOL | | | | | | 1 AND 4 mm | |
| | SOLUTION | | GEL | | OTHER | | | % of |
| Patch | refer- | | refer- | | COMPONENT | | Trans- | solubilised |
| No. | ence | % | ence | % | Type | % | parency | menthol |
| 1 | Solution A | 27 | 1 | 73 | | | Yes | 13.5 |
| 2 | Solution A | 17 | 1 | 77 | IPM | 6 | Yes | 8.5 |
| 3 | Solution A | 10 | 1 | 80 | IPM | 10 | Yes | 5 |
| 4 | | | 1 | 84 | IPM | 16 | Yes | 0 |
| 5 | Solution B | 30 | 1 | 70 | | | Yes | 15 |
| 6 | | | 1 | 70 | D5 | 30 | Yes | 0 |
| 7 | Solution A | 30 | 2 | 70 | | | Yes | 15 |

24 hours after they had been prepared, the patches were transparent; they were also self-adhering to skin, glass, and plastics materials, and released menthol which could be detected owing to its specific odour.
These characteristics were maintained over time. Exposed to contact with air, at ˜20-23° C., the menthol odour was still perceptible after more than a week. They also maintained their self-adhesion and transparency.

It will be understood that the invention defined by the appended claims is not limited to the specific embodiments indicated in the foregoing description but includes variations thereof departing neither from the scope nor from the spirit of the present invention.

1-12. (canceled)

13. A silicone material, optionally an adhesive material, for releasing an active molecule useful in cosmetics or in personal care or a pharmaceutically or biologically active molecule for pharmaceutical application, which is formed by an optionally adhesive, silicone material wherein there is incorporated said molecule and a compatibility agent which is miscible with the silicone components of the silicone material and wherein said active molecule is soluble, said compatibility agent being selected from the group consisting of isopropyl myristate, isopropyl palmitate, isononyl isononanoate, neopentyl glycol dioctanoate, branched paraffins, organofunctional silicones, and a silicone oil comprising a cyclic chain of 4, 5, 6 or 7 D siloxyl units of formula:
(R)₂SiO_{2/2}
wherein the symbols R, which are the same or different, each represent a linear or branched C₁-C₆ alky radical, or an aryl or alkylaryl radical having 6 to 8 carbon atoms.

14. The silicone material as defined by claim 13, wherein the compatibility agent content is from 5% to 50% by weight, expressed relative to the total composition.

15. The silicone material as defined by claim 13, wherein the concentration of solubilized active material is at least 1.5 times greater than the maximum concentration which it would be possible to solubilize in the absence of the compatibility agent.

16. The silicone material as defined by claim 13, wherein the amount of incorporated active molecule exceeds the solubilization capacity of the compatibility agent such that the active molecule is present in the solubilized and dispersed state.

17. The silicone material as defined by claim 13, wherein the total content of active molecule and compatibility agent is up to 75% by weight of the total composition.

18. The silicone material as defined by claim 13, wherein the concentration of active molecule solubilized in the silicone material is greater than or equal to 5, 10, 15, 20, 25 or 30% by weight based on the total weight of the material.

19. The silicone material as defined by claim 13, wherein the total active molecule content is from 5% to 60% by weight based on the total weight of the material.

20. The silicone material as defined by claim 13, wherein the silicone material is obtained by a hydrosilylation reaction between a polyorganosiloxane carrying alkenylsiloxy units and a polyorganosiloxane carrying hydrogenosiloxy units in the presence of a hydrosilylation catalyst.

21. The silicone material as defined by claim 13, in gel or elastomer form.

22. A shaped article comprising the silicone material as defined by claim 13.

23. A shoe, a knee pad, a clothing, sports or bodily protection article, or any item to be worn by a living being, comprising the silicone material as defined by claim 13.

24. The silicone material as defined by claim 13, wherein the active molecule is selected from the group consisting of methanol, methyl diisopropyl propionamide and mixtures thereof.
